(19) United States
(12) Patent Application Publication
(10) Pub. No.: US 2026/0134995 A1
(43) Pub. Date: May 14, 2026

(54) ANALYSIS METHOD FOR SUBJECT'S SAMPLE BASED ON DE NOVO STRUCTURAL VARIATION AND HARDWARE APPARATUS

(71) Applicant: IUCF-HYU (INDUSTRY-UNIVERSITY COOPERATION FOUNDATION HANYANG UNIVERSITY), Seoul (KR)

(72) Inventors: Jin-Wu NAM, Seoul (KR); Hyun-Woo KIM, Yongin-si (KR)

(73) Assignee: IUCF-HYU (INDUSTRY-UNIVERSITY COOPERATION FOUNDATION HANYANG UNIVERSITY), Seoul (KR)

(21) Appl. No.: 19/384,105

(22) Filed: Nov. 10, 2025

(30) Foreign Application Priority Data
Nov. 11, 2024 (KR) 10-2024-0159596

(51) Int. Cl. G16H 50/20; G16B 20/20; G16B 30/10; G16H 15/00; G16H 50/70

(52) U.S. Cl. CPC G16H 50/20; G16B 20/20; G16B 30/10; G16H 15/00; G16H 50/70

(57) ABSTRACT

A method for detecting de novo structural variations includes: a genome analysis apparatus extracting k-mer data of a target individual from genome sequencing data of a target individual. The method may include: extracting k-mer data of a target individual from genome sequencing data, comparing a reference-genome k-mer database—including parents' and pan-genome k-mers—with the target individual's k-mers to select target-individual-specific k-mers, determining target-individual-specific reads, identifying candidate de novo structural-variation regions, predicting discordant read pairs using a machine-learning model, selecting final de novo regions based on an estimated variant allele frequency, and generating a clinical report.

### CROSS REFERENCE TO RELATED APPLICATIONS

The present application claims the benefit of and priority to Korean Patent Application No. 10-2024-0159596, filed Nov. 11, 2024, the entire contents of which are incorporated herein by reference for all purposes.

### BACKGROUND

### Technical Field

The present disclosure relates to a technique for detecting de novo structural variations in samples.

### Description of the Related Art

De novo structural variations are genetic variations that appear for the first time in offspring but are not found in the somatic cells of the parents. De novo structural variations are likely to be the cause of phenotypes (such as diseases) that appear specifically in the individual. Conventional de novo structural variation detection has been performed by mapping sequencing data from samples derived from parents and children to a reference genome to detect structural variations, and then excluding structural variations shared by parents and children.

Conventional de novo structural variation detection techniques analyze parents and child (a trio) data separately, which makes the analysis complex and time-consuming.

The description of the related art should not be assumed to be prior art merely because it is mentioned in or associated with this section. The description of the related art includes information that describes one or more aspects of the subject technology, and the description in this section does not limit the invention.

### SUMMARY

In one or more aspects of the present disclosure, a method for analyzing a sample of a subject based on de novo structural variation, includes: acquiring genome sequencing data of a target individual; extracting k-mer data from the sequencing data; comparing a reference genome k-mer database—including parental and pan-genome k-mer data—with the individual's k-mer data to select child-specific k-mers; determining child-specific paired-end reads; identifying candidate regions of de novo structural variations; predicting, for each candidate region, a number of discordant read pairs; estimating variant allele frequency for each candidate region; selecting final de novo structural variation regions based on the estimated values; and generating a diagnostic report.

In one or more aspects of the present disclosure, a hardware apparatus for detecting de novo structural variations, including: an interface device that receives genome sequencing data of a child who is an analysis target and genome sequencing data of parents of the child; a storage device that stores a prediction model that predicts the number of discordant read pairs in a de novo structural variation candidate region in order to estimate the variant allele frequency of the candidate region; and a processor that extracts k-mer data of the child from the genome sequencing data of the child, extracts k-mer data of the parents from the genome sequencing data of the parents, compares a reference genome k-mer database including the k-mer data of the child's parents with the k-mer data of the child to select child-specific k-mers, determines de novo structural variation candidate regions based on child-specific sequencing reads determined based on the child-specific k-mers, and selects a final de novo structural variation region by removing noise from each of the de novo structural variation candidate regions.

Additional features, advantages, and aspects of the present disclosure are set forth in part in the description that follows and in part will become apparent from the present disclosure or may be learned by practice of the inventive concepts provided herein. Other features, advantages, and aspects of the present disclosure may be realized and attained by the descriptions provided in the present disclosure, or derivable therefrom, and the claims hereof as well as the drawings. It is intended that all such features, advantages, and aspects be included within this description, be within the scope of the present disclosure, and be protected by the following claims. Nothing in this section should be taken as a limitation on those claims. Further aspects and advantages are discussed below in conjunction with embodiments of the present disclosure.

It is to be understood that both the foregoing description and the following description of the present disclosure are examples, and are intended to provide further explanation of the disclosure as claimed.

### BRIEF DESCRIPTION OF THE DRAWINGS

The accompanying drawings, which are included to provide a further understanding of the present disclosure, are incorporated in and constitute a part of this present disclosure, illustrate aspects and embodiments of the present disclosure, and together with the description serve to explain principles and examples of the disclosure. In the drawings:

FIG. 1 illustrates an example of a system for detecting de novo structural variations.

FIG. 2 illustrates an example of a process for detecting de novo structural variations.

FIG. 3 illustrates an example of a filtering process for removing false positive somatic structural variations in the de novo structural variation detection process.

FIGS. 4A-4C illustrate an example of analysis results predicted using a prediction model.

FIG. 5 shows the results of predicting de novo structural variations in the conventional technique and the proposed technique.

FIGS. 6A-6B show the results of comparing the time required for the de novo structural variation prediction process in the conventional technique and the proposed technique.

FIG. 7 illustrates an example of a hardware device for detecting de novo structural variations.

Throughout the drawings and the detailed description, unless otherwise described, the same drawing reference numerals should be understood to refer to the same elements, features, and structures. The sizes of regions and elements, and depiction thereof may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of,” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements may represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements may represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element may represent (i) a portion (or a part) of the element, (ii) one or more portions (or parts) of the element, or (iii) the element, or all portions of the element.

The expression of a first element, a second elements “and/or” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings.

Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

Structural variation includes insertions-deletions (Indels), copy number variation (CNV), inversions, translocations, genetic variations of 50 bp (base pair) or more, duplications, etc. Structural variations are usually longer than single nucleotide polymorphisms (SNPs) and shorter than chromosomal abnormalities.

Structural variation detection may be performed through Next Generation Sequencing (NGS) analysis. NGS analysis includes single-end library and paired-end library methods. The paired-end technique is a method of mapping and comparing both end reads (forward read and reverse read) of sample genome data to reference genome data.

Sample sequence data means genome sequence data of a sample to be analyzed. Sample sequence data may be sequence data generated through NGS. In this case, the sample may include a sample of a child and a sample of the child's parents for de novo structural variation analysis.

Reference genome or reference genome data means genome sequence data that serves as a comparison target for variation analysis of sample sequence data. Reference genome data may basically be prepared for each race. For example, reference genome data may be data such as hg38, HuRef, NA12878, KOREF, etc. Furthermore, reference genome data may be composed of reference genome data from multiple races.

In the following description, it is explained that a genome analysis apparatus performs de novo structural variation detection. The genome analysis apparatus may be implemented in various forms. For example, the genome analysis apparatus may be implemented as a PC, a server on a network, a smart device, a chipset embedded with a dedicated program, etc.

The analysis target of de novo structural variation is a child individual. Hereinafter, the analysis target is referred to as a child or individual. Individual means not only humans but also animals. Parent-child trio or trio refers to the child who is the analysis target and the child's parents.

De novo structural variations may contribute to genetic diseases. Genetic diseases resulting from de novo structural variations include neuromuscular diseases (Duchenne muscular dystrophy, Spinal muscular atrophy, etc.), neurodevelopmental disorders, congenital heart defects, rare genetic syndromes (Williams-Beuren syndrome, Smith-Magenis syndrome, etc.).

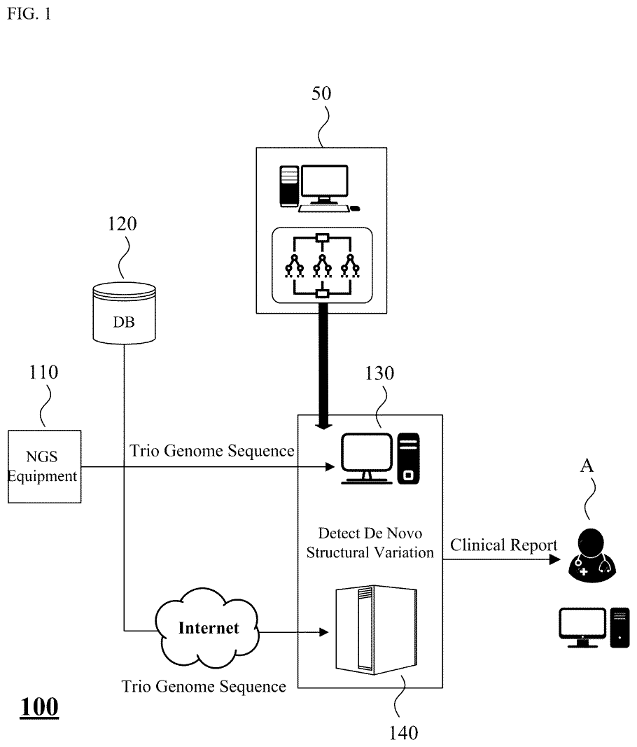

FIG. 1 illustrates an example of a system (100) for detecting de novo structural variations. FIG. 1 shows an example where the genome analysis apparatus is a computer terminal (130) and a server (140).

The NGS equipment (110) sequences a sample's genome. The NGS equipment (110) may perform Whole Genome Sequencing (WGS), etc. The NGS equipment (110) may generate genome sequence data by sequencing genome data for child and parents. The NGS equipment (110) may generate trio genome sequence data.

The database (DB, 120) may store trio genome sequence data of samples.

The training device (50) constructs a machine learning model to be used for removing somatic structural variations corresponding to noise in the process of detecting de novo structural variations in children. The machine learning model is a model that predicts discordant read pairs for estimating the variant allele frequency of child samples. Hereinafter, the model that predicts the number of discordant read pairs and estimates the variant allele frequency is referred to as a prediction model. The process of constructing the prediction model will be described later.

The computer terminal (130) receives trio genome sequence data from the NGS equipment (110) or DB (120). The computer terminal (130) may extract k-mers from the child's genome sequencing data, and compare the child's k-mers with a reference-genome k-mer database including the parents' and pan-genome k-mers to determine child-specific k-mers. The computer terminal (130) may determine de novo structural variation candidates based on child-specific k-mers. The computer terminal (130) may predict final de novo structural variations by removing (filtering) somatic structural variations among de novo structural variation candidates using the prediction model. The computer terminal (130) may output (provide) the analysis results to user A.

The server (140) receives trio genome sequence data from the NGS equipment (110) or DB (120). The server (140) may extract k-mers of the child from the child genome sequence, and compare the child's k-mers with reference genome k-mers including parents to determine child-specific k-mers. The server (140) may determine de novo structural variation candidates based on child-specific k-mers. The server (140) may predict final de novo structural variations by removing (filtering) somatic structural variations among de novo structural variation candidates using the prediction model. The server (140) may transmit the analysis results to the terminal of user A.

The computer terminal (130) and server (140) are hardware devices that process data streams received from the NGS equipment (110).

The computer terminal (130) and/or server (140) may generate a clinical report or diagnostic report including clinical information according to the de novo structural variations of the subject. The clinical report may include disease occurrence risk based on the identified de novo structural variations.

The computer terminal (130) and/or server (140) may store the analysis results in the DB (120).

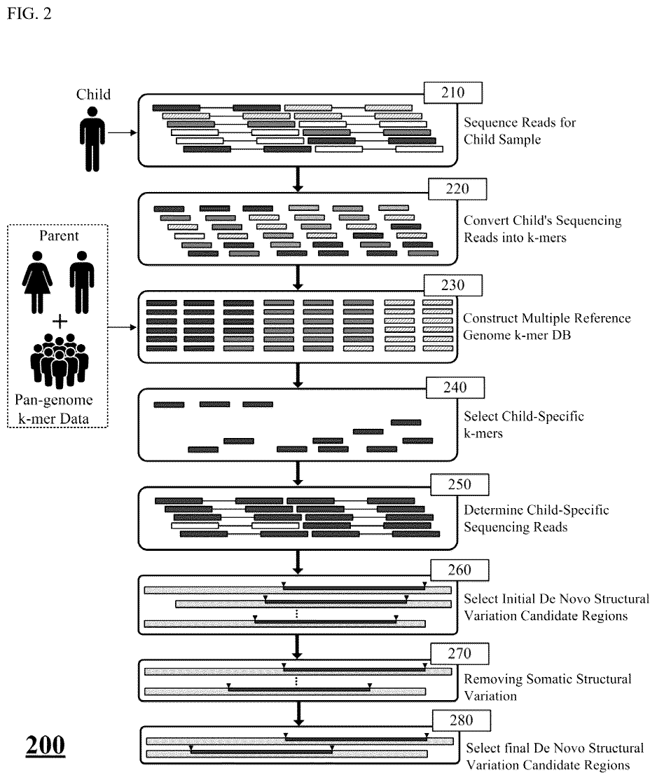

FIG. 2 illustrates an example of a process (200) for detecting de novo structural variations.

Before detecting de novo structural variations, the genome analysis apparatus acquires genome sequencing data. At this time, the genome sequencing data may include genome sequence data of parents and children. The genome sequencing data may be a WGS dataset for parents and child samples. The genome sequencing data may be genome data obtained by sequencing the genome data of the trio using a paired-end library. That is, the genome sequence data may be composed of sequencing reads for samples.

The genome analysis apparatus receives sequencing reads for child samples (210). Sequencing reads may consist of paired-end reads (forward and reverse).

The genome analysis apparatus may convert the child's sequencing reads into k-mers (e.g., 31-mers, k=31) (220).

The genome analysis apparatus may generate the child's 31-mers using a tool for conventional k-mer data processing. Researchers extracted 31-mers using the KMC tool (Marek Kokot et al., “KMC 3: counting and manipulating k-mer statistics.” Bioinformatics 33.17 (2017): 2759-2761. reference). Through this, the genome analysis apparatus may extract the child's 31-mer data from the child's sequencing reads. In some cases, the length k of k-mer may be set to a different value. Hereinafter, description will be made based on 31-mer.

The genome analysis apparatus also receives sequencing reads for parents' samples. The genome analysis apparatus may convert each sequencing read of the parents into k-mer (31-mer) data of 31 bp length. The genome analysis apparatus may merge paternal 31-mer data and maternal 31-mer data using the KMC tool (ocsum parameter) to generate 31-mer data of the parents.

The genome analysis apparatus may integrate the parents' 31-mer data with other pan-genome 31-mer data to construct a multiple reference genome 31-mer DB (230). The genome analysis apparatus may construct a multiple reference genome 31-mer DB by combining the parents' 31-mer data with pan-genome 31-mer data using the KMC tool.

Pan-genome k-mer (31-mer) data may include multiple reference genomes. Researchers used pan-genome k-mer data including 894 human genome assemblies and 8 human reference genomes.

The genome analysis apparatus may select child-specific 31-mer(s) by comparing the child's 31-mer data with the multiple reference genome 31-mer DB (240). At this time, the genome analysis apparatus may select child-specific 31-mer(s) using conventionally studied techniques. Researchers used the k-mer filtering technique ETCHING (Jang-il Sohn et al. “Ultrafast prediction of somatic structural variations by filtering out reads matched to pan-genome k-mer sets.” Nature Biomedical Engineering 7.7 (2023): 853-866. reference) to select child-specific 31-mer(s) based on the multiple reference genome 31-mer DB.

The genome analysis apparatus may determine sequencing reads containing the corresponding 31-mer based on the child-specific 31-mer(s) (250). Through this process, the genome analysis apparatus may determine child-specific sequencing reads.

The genome analysis apparatus may select initial de novo structural variation(s) candidate regions from the child's sequence based on the child-specific sequencing reads (260). The genome analysis apparatus may determine the sequence region defined by the reads as a de novo structural variation candidate region based on the child-specific sequencing reads (forward read and reverse read). The genome analysis apparatus may determine multiple initial de novo structural variation candidate regions.

The genome analysis apparatus may remove (filter) somatic structural variations region(s) from the initial de novo structural variation candidate regions (270). The genome analysis apparatus may use the filtered de novo structural variation candidate region(s) as the final de novo structural variation analysis target.

Meanwhile, in the filtered de novo structural variation candidate regions, there is a possibility that germline structural variations shared with parents may be incorrectly detected as de novo structural variations due to SNPs existing near the structural variations. At this time, the genome analysis apparatus may detect structural variations of parents (paternal and maternal) using the reference genome k-mer DB. At this time, the reference genome k-mer DB may be composed of race-specific reference genomes that do not include k-mer data of the parents. The genome analysis apparatus may detect parent-specific structural variations separately from (in parallel with or in advance of) the child-specific structural variation detection process.

The genome analysis apparatus may select the final de novo structural variation candidate region by removing structural variation(s) shared with parents, which is separatedly detected in the parents using the reference genome k-mer DB that do not include k-mer data of the parents, from the filtered de novo structural variation candidate regions (280).

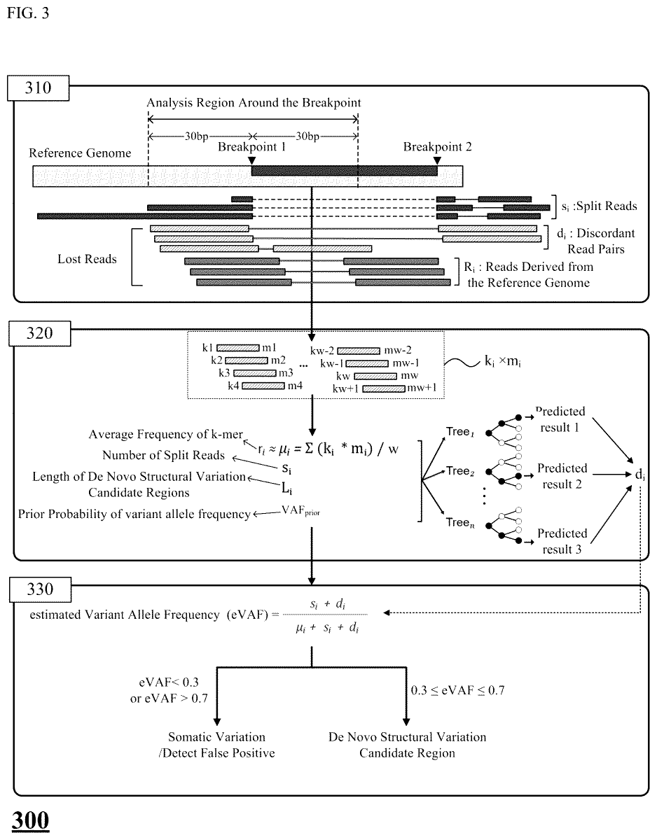

FIG. 3 illustrates an example of a filtering process (300) for removing false positive somatic structural variations in the de novo structural variation detection process. The genome analysis apparatus performs a filtering process for each of the de novo structural variation candidate regions. FIG. 3 is an example of a filtering process for one de novo structural variation candidate region. FIG. 3 assumes a filtering process for de novo structural variation candidate region i among multiple de novo structural variation candidate regions.

The genome analysis apparatus may perform filtering on de novo structural variation candidate region i using k-mers.

The genome analysis apparatus determines an analysis region (window) in de novo structural variation candidate region i. The analysis region may be determined based on the breakpoint that defines de novo structural variation candidate region i. The analysis region may be a 61 bp length region (region around the breakpoint) from a 30 bp length region before the breakpoint to a 30 bp length region after the breakpoint.

The genome analysis apparatus may determine the analysis region based on at least one of the two breakpoints (breakpoint 1 and breakpoint 2) that define de novo structural variation candidate region i. That is, the genome analysis apparatus may remove somatic-derived structural variations for de novo structural variation candidate region i through analysis of the analysis region determined based on any one of the two breakpoints. FIG. 3 is an example of filtering for the analysis region determined based on breakpoint 1.

The genome analysis apparatus converts the sequence of the reference genome belonging to the analysis region of de novo structural variation candidate region i into 31-mers.

The genome analysis apparatus calculates information necessary for estimating the variant allele frequency for child sequencing reads using reference genome 31-mer data mapped to de novo structural variation candidate region i (310).

The variant allele frequency may be defined as the ratio of reads showing variation among all reads mapped to de novo structural variation candidate region i. Reads showing variation may include split reads and discordant read pairs among child sequencing reads.

First, the genome analysis apparatus may calculate the number sᵢ of split reads in de novo structural variation candidate region i. A split read is a read that is separated when mapped to the reference genome, with some parts being mapped to different regions among child sequencing reads. In this case, the split read refers to a split read that exactly corresponds to the breakpoint among child-specific reads.

Discordant read pairs refer to reads that are mapped at different positions in the reference genome or have different mapping directions for the same child sequencing read pair. However, the number di of discordant read pairs cannot be calculated from the mapped read data because it is lost in the k-mer filtering process of the genome analysis apparatus.

The genome analysis apparatus may predict the number of reads derived from the reference genome based on k-mers. The genome analysis apparatus converts the sequence of the reference genome located in the analysis region into 31-mers. At this time, the reference genome means the multiple reference genome constructed in step 230. The genome analysis apparatus may estimate the frequency of the reference genome's 31-mer sequence using the child's 31-mer information.

The genome analysis apparatus may use the average value of all 31-mers located in the analysis region to predict the depth of coverage (DOC) of the reference genome at that position. The number rᵢ of reads derived from the reference genome in de novo structural variation candidate region i approximates the average frequency μᵢ of reference genome 31-mers located in the analysis region of de novo structural variation candidate region i. Therefore, the genome analysis apparatus may perform analysis by replacing the number rᵢ of reads derived from the reference genome with Hi. The average frequency μᵢ of reference genome 31-mers is as shown in Equation 1 below.

$${r}_{i} \approx {μ}_{i} = \frac{{\sum }_{i}^{n} ( {k}_{i} \times {m}_{i} )}{{w}_{i}} \qquad [ Equation 1 ]$$

μᵢ is the average k-mer frequency of the reference genome in the analysis region of de novo structural variation candidate region i. kᵢ is the k-mer (31-mer) frequency of the input sample, and mᵢ is the mappability score of the reference genome. n is the number of k-mers. wᵢ is the length of the window (analysis region).

FIG. 3 shows values (kᵢ×mᵢ) obtained by multiplying the k-mer frequency and the mappability score at each base position in a window (analysis region) of size w. The mappability score may be determined according to each base position of the sequence. The mappability score of the reference genome may be calculated through tools such as GenMAP (Pockrandt, Christopher, et al. “GenMap: ultra-fast computation of genome mappability.” Bioinformatics 36.12 (2020): 3687-3692. reference).

The genome analysis apparatus may estimate the number of discordant read pairs using the aforementioned prediction model (320).

The prediction model may be any one of various types of models. The prediction model may be any one of machine learning models. There are various types of machine learning models. For example, machine learning models include decision trees, random forest (RF), K-nearest neighbor (KNN), Naive Bayes, support vector machine (SVM), artificial neural network (ANN), regression models, etc. Deep learning network (DNN) is one of the representative ANN models. Researchers constructed a prediction model in RF form. The model construction process and verification results will be described later.

The prediction model is trained using training data. Training data consists of input data and label values. The input data includes the average frequency u of all reads (k-mers), the length L of the de novo structural variation candidate region, the number S of split reads that exactly correspond to the breakpoint among child-specific reads, and the prior probability of variant allele frequency VAFₚᵣᵢₒᵣ. VAFₚᵣᵢₒᵣ is a variation probability value for detecting variation candidates. VAFₚᵣᵢₒᵣ may be calculated based on the number of split reads that are known variations in the current de novo structural variation candidate region. VAF_{prior_i} for de novo structural variation candidate region i may be defined as shown in Equation 2 below.

$${VAF}_{p {rior}_{i}} = \frac{{w}_{i} \times {s}_{i}}{{\sum }_{j}^{n} ( {k}_{j} \times {m}_{j} ) + {w}_{i} \times {s}_{i}} \qquad [ Equation 2 ]$$

At this time, j means the j-th k-mer among n k-mers in de novo structural variation candidate region i. kⱼ is the frequency of the j-th k-mer among n k-mers, and mⱼ is the mappability score of the j-th k-mer among n k-mers.

The trained prediction model receives μᵢ in de novo structural variation candidate region i, the length Li of candidate region i, the number of split reads Sᵢ, and the prior probability of variant allele frequency VAF_{prior_i} as input and outputs the number di of discordant read pairs for the de novo structural variation candidate region i.

The genome analysis apparatus may estimate the variant allele frequency and filter false positives in the current de novo structural variation candidate region based on the estimated variant allele frequency (330).

The estimated variant allele frequency eVAFᵢ in de novo structural variation candidate region i may be defined as shown in Equation 3 below.

$${eVAF}_{i} = \frac{{s}_{i} + {d}_{i}}{{μ}_{i} + {s}_{i} + {d}_{i}} \qquad [ Equation 3 ]$$

(i) If eVAFᵢ<0.3 (lower threshold), the genome analysis apparatus may determine the de novo structural variation candidate region i as a somatic variation and remove it from the de novo structural variation candidate group. If eVAFᵢ>0.7 (upper threshold), the genome analysis apparatus may determine it as an error and remove the de novo structural variation candidate region i from the de novo structural variation candidate group.
(ii) If 0.3≤eVAFᵢ≤0.7, the genome analysis apparatus may select the de novo structural variation candidate region i as the final de novo structural variation. At this time, the selected de novo structural variation may not be a definitive de novo structural variation but a candidate with a very high possibility of de novo structural variation.

Meanwhile, at this time, different threshold values may be applied. For example, the genome analysis apparatus may use 0.2 as the lower threshold and 0.8 as the upper threshold to more broadly select final de novo structural variation candidates.

The following describes the de novo structural variation detection process and noise filtering model construction process performed by researchers.

Researchers constructed a prediction model in RF form using a training dataset. The prediction model in RF form constructs each tree using bootstrapped samples of the training dataset during the learning process. In the inference process, the RF model may determine the final predicted value by averaging the predicted values of each decision tree.

The training dataset was constructed using synthetic datasets using a structural variation simulation program (Ewing, Adam D., et al. “Combining tumor genome simulation with crowdsourcing to benchmark somatic single-nucleotide-variant detection.” Nature methods 12.7 (2015): 623-630. reference).

Researchers verified the performance of the model constructed using real datasets and synthetic datasets, as well as de novo structural variation performance.

The actual verification dataset used the Ashkenazim Jewish dataset generated from a genome project conducted by the Genome In A Bottle (GIAB) consortium. The dataset includes short reads from Whole Genome Sequencing (WGS) and long read data from Circular Consensus Sequencing (CCS) for Ashkenazim Jewish trios. The Ashkenazim Jewish trio includes a structural variation set extracted from HG002 child's data of Ashkenazim Jews and parents' data extracted from HG003 and HG004. Meanwhile, low-quality reads were removed from the dataset, and duplicate reads were also removed by mapping to reference genomes (hg19 or hg38).

In addition, researchers also constructed a synthetic dataset for verification. Researchers constructed paired-end WGS datasets with reads of 150 bp length and fragments of 500 bp length for hg38 using the NGSNGS (Henriksen, Rasmus Amund, Lei Zhao, and Thorfinn Sand Korneliussen. “NGSNGS: next-generation simulator for next-generation sequencing data.” Bioinformatics 39.1 (2023): btad041. reference) tool as verification data. Structural variation regions were randomly selected in the range of 50 bp to 5 kb size. The selected structural variation regions were randomly assigned VAF, with somatic structural variations set in the range of 0.1 to 0.29 and de novo structural variations set in the range of 0.3 to 0.7. The final variation region data combined the selected structural variation regions for trio analysis and a set of 500 common structural variations with population allele frequency (PAF) of 0.01 or more extracted from dbVAR. The final verification data was prepared by inserting the generated final variation regions into the reference genome data.

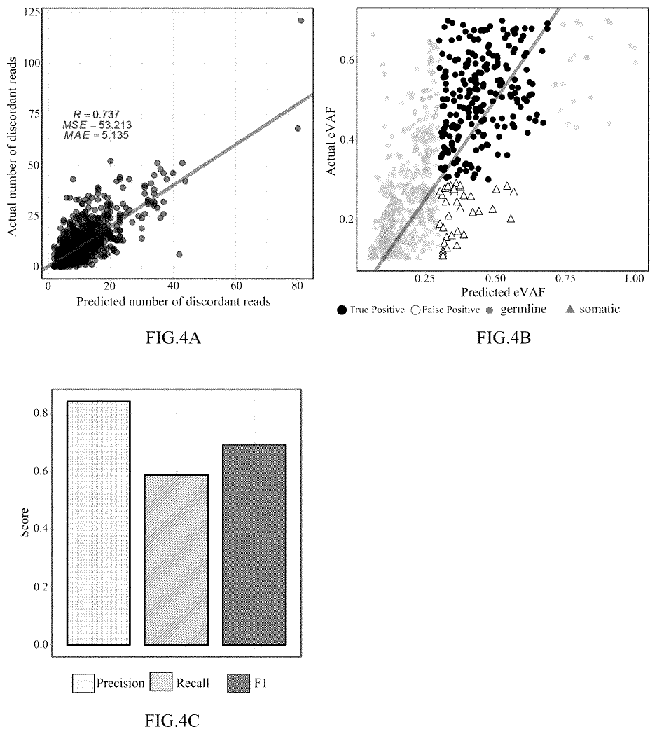

FIGS. 4A-4C illustrates an example of analysis results predicted using a prediction model.

FIG. 4A is the predicted number of discordant reads using an individual verification set composed of 696 virtual breakpoints. The Pearson correlation coefficient R between actual discordant reads and predicted discordant reads showed results exceeding 0.7.

FIG. 4B is eVAF predicted using the predicted number of discordant reads. FIG. 4B shows prediction results for the individual verification set composed of 696 virtual breakpoints in FIG. 4A. FIG. 4B shows prediction results for one window (analysis region). FIG. 4C shows the performance of eVAF prediction. The eVAF prediction performance showed an F1 score of 0.7.

That is, the number of discordant reads predicted by the prediction model and the eVAF predicted using them also showed considerable performance.

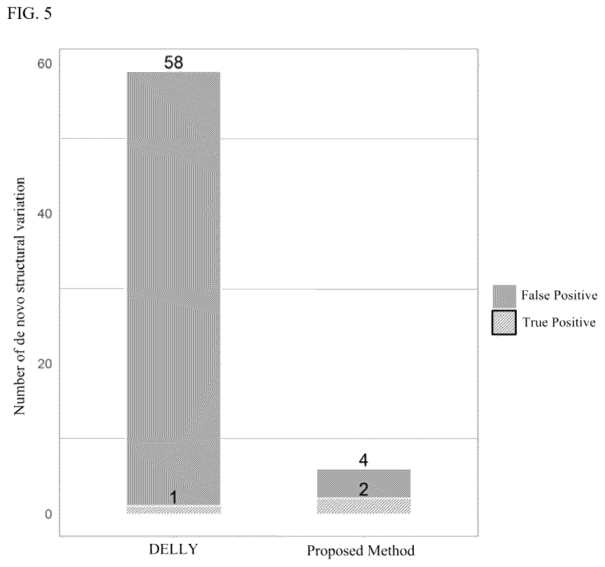

FIG. 5 shows the results of predicting de novo structural variations in the conventional technique and the proposed technique. The conventional technique used DELLY (Rausch, Tobias, et al. “DELLY: structural variant discovery by integrated paired-end and split-read analysis.” Bioinformatics 28.18 (2012): 1333-i339. reference). The proposed technique refers to the de novo structural variation detection technique described in FIGS. 2 and 3. FIGS. 4A-4C show the results of analysis using actual HG002 Jewish trio data. It was confirmed that the proposed technique has 93% fewer false positives compared to DELLY. In addition, it was confirmed that the proposed technique showed higher recall by predicting one case that was determined as a false negative in DELLY's results as a true positive.

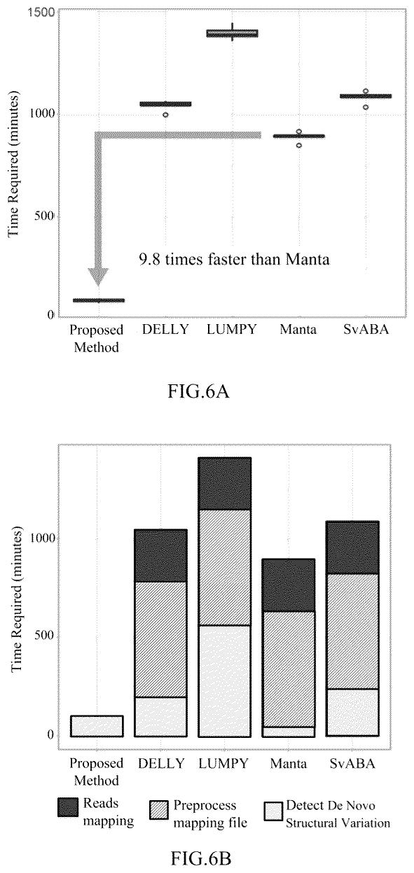

FIGS. 6A-6B shows the results of comparing the time required for the de novo structural variation prediction process in the conventional technique and the proposed technique. The conventional techniques used DELLY, Lumpy (Layer, Ryan M., et al. “LUMPY: a probabilistic framework for structural variant discovery.” Genome biology 15 (2014): 1-19. reference), Manta (Chen, Xiaoyu, et al. “Manta: rapid detection of structural variants and indels for germline and cancer sequencing applications.” Bioinformatics 32.8 (2016): 1220-1222. reference), and SvABA (Wala, Jeremiah A., et al. “SvABA: genome-wide detection of structural variants and indels by local assembly.” Genome research 28.4 (2018): 581-591. reference).

FIGS. 6A-6B shows the results of repeating the de novo structural variation detection process 5 times using the proposed technique and conventional techniques. FIG. 6A illustrate the total prediction time for de novo structural variation itself, and FIG. 6B illustrate the time required for each process of de novo structural variation prediction. Because the proposed technique does not perform the mapping process, the analysis time was shorter than conventional techniques. The proposed technique showed analysis time about 9.8 times faster than Manta, which showed relatively fast analysis time among conventional techniques.

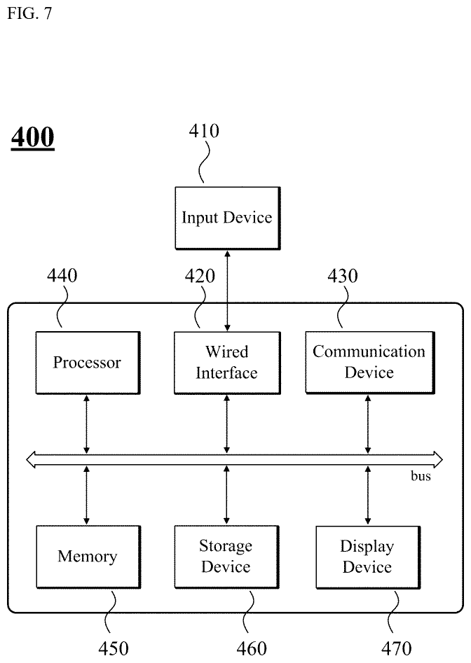

FIG. 7 illustrates an example of a hardware device (400) for detecting de novo structural variations.

The hardware device (400) corresponds to the genome analysis apparatus described above (130 and 140 in FIG. 1). Alternatively, the hardware device (400) may be a device that interprets de novo structural variations to generate a certain clinical report.

The hardware device (400) may be a device that processes data streams generated by NGS equipment in real time. At this time, the hardware device (400) may be a system integrated with NGS equipment.

The hardware device (400) may be physically implemented in various forms. For example, the hardware device (400) may have the form of a computer device such as a PC, a network server, a dedicated chipset for data processing, FPGA, etc.

The hardware device (400) may include an input device (410), a wired interface (420), a communication device (430), a processor (440), a memory (450), and a storage device (460).

In addition, the hardware device (400) may include an input device (410), a wired interface (420), a communication device (430), a processor (440), a memory (450), a storage device (460), and a display device (470).

Each internal component of the hardware device (400) may be connected by a bus. A specific bus may be used depending on the type of entity being connected. For example, the bus may be any one of AMBA (AHB/AXI/APB), PCIe, Serial Peripheral Interface (SPI), or Mobile Industry Processor Interface (MIPI).

The input device (410) is a device that receives user commands or necessary data.

The input device (410) may receive genome sequencing data of a child (subject) and genome sequencing data of the child's parents.

The input device (410) may receive pan-genome data.

The input device (410) may be any one of various types of devices. For example, the input device (410) may be at least one of a mouse, keyboard, touch input device, camera, Small Computer System Interface (SCSI) device, Peripheral Component Interconnect (PCI) bus-based device, or ATA Packet Interface (ATAPI) device.

Furthermore, the input device (410) may be NGS equipment.

The wired interface (420) is a device component that transmits data transmitted by the input device (410) into the device. The wired interface (420) may be composed of a software driver and hardware.

The wired interface (420) may include a controller corresponding to each input device, a device driver that controls the operation of the controller, and a kernel I/O subsystem that comprehensively manages input/output control requests of the device driver. The kernel I/O subsystem stores input/output requests from the device driver in a queue and schedules the requests based on request priority or device status.

The wired interface (420) may include interfaces such as PS/2, Universal Serial Bus (USB), Ethernet port, HDMI, MIPI CSI, DisplayPort, Thunderbolt, etc.

The communication device (430) means a component that receives and transmits certain information through an external wired or wireless network. The communication device (430) may be composed of circuits including an antenna and a communication module (S/W module, chip, etc.) corresponding to a communication protocol. The communication protocol may be at least one of wired LAN (Ethernet), wireless LAN (IEEE 802.11), mobile communication (LTE, 5G NR, etc.), Bluetooth, NFC, etc.

The communication device (430) may receive genome sequencing data of a child and genome sequencing data of the child's parents from an external object.

The communication device (430) may receive pan-genome data from an external object.

The communication device (430) may transmit information about the child's de novo structural variation region or de novo structural variation candidate region calculated as analysis results to an external object such as a user terminal.

The communication device (430) may transmit a clinical report generated based on de novo structural variations to an external object.

The processor (440) controls the operation of all components of the hardware device (400).

The processor (440) may perform operations on at least one application or computer program for executing methods/operations according to various embodiments of the present disclosure.

The processor (440) is a general-purpose processor that executes at least some of the control programs installed in the storage device (460) or at least some of the programs loaded in the memory (450).

The processor (440) may be implemented as circuitry such as a system on chip (SoC) or an integrated circuit (IC).

The processor (440) may include one or more processors. For example, the processor (440) may include a combination of one or more processors such as a central processing unit (CPU), microprocessor unit (MPU), micro controller unit (MCU), graphic processing unit (GPU), neural processing unit (NPU), digital signal processor (DSP), application processor (AP), communication processor (CP), or any form of processor well known in the technical field of the present disclosure.

The memory (450) may store data generated during the de novo structural variation detection and de novo structural variation-based subject evaluation or diagnosis process. The memory (450) is volatile memory such as DRAM or SRAM.

The storage device (460) may store a machine learning model (prediction model) that predicts the number of discordant read pairs.

The storage device (460) may store various programs or tools used for genome data search and processing.

The storage device (460) may store genome sequencing data of the child who is the analysis target and genome sequencing data of the child's parents.

The storage device (460) may store pan-genome data for use in creating multiple reference genomes. At this time, the pan-genome data may be k-mer data.

The storage device (460) may store detected de novo structural variation candidates or de novo structural variation regions.

The storage device (460) may store a diagnostic report based on de novo structural variations.

The storage device (460) may be implemented as a device such as a hard disk drive, Solid State Drive, USB flash drive, memory card, optical disk, or network-based storage device (Network Attached Storage, cloud storage, etc.).

The display device (470) may output an interface screen for the analysis process, analysis results, etc.

The display device (470) may be implemented as various types of devices.

The display device (470) may be implemented in various display methods such as liquid crystal, plasma, light-emitting diode, organic light-emitting diode, surface conduction electron-emitter, carbon nano-tube, nano-crystal, etc.

The processor (440) may preprocess the genome sequencing data of the child and the genome sequencing data of the parents to a certain extent.

The processor (440) may extract k-mer data from the genome sequencing data of the child.

The processor (440) may extract k-mer data from the paternal genome sequencing data. The processor (440) may extract k-mer data from the maternal genome sequencing data. The processor (440) may generate k-mer data of parents by combining paternal k-mer data and maternal k-mer data. Furthermore, the processor (440) may generate a multiple reference genome k-mer database by combining k-mer data of the parents and pan-genome k-mer data. At this time, pan-genome k-mer data may be extracted from genome sequencing data of multiple objects or extracted from reference genomes.

The processor (440) may select child-specific k-mers by comparing the multiple reference genome k-mer database with the child's k-mer data. The processor (440) is a component that compares k-mers. The processor (440) may determine child-specific sequencing reads to which the k-mer belongs based on child-specific k-mers.

The processor (440) may select de novo structural variation candidate regions based on child-specific sequencing reads.

The processor (440) may filter regions corresponding to noise among de novo structural variation candidate regions. The filtering process is as described in FIG. 3.

Description will be made based on one de novo structural variation candidate region i.

The processor (440) determines an analysis region (window) based on the breakpoint of de novo structural variation candidate region i.

The processor (440) converts reference genome data located in the analysis region into k-mers. Thereafter, the processor (440) performs a filtering process based on the reference genome k-mers.

The processor (440) determines split reads that exactly correspond to the two breakpoints of de novo structural variation candidate region i (that is, as shown in FIG. 3, one end of the split read matches breakpoint 1 and the other end of the split read matches breakpoint 2).

The processor (440) calculates the average frequency μᵢ of reference genome k-mers located in the analysis region of de novo structural variation candidate region i. Reference genome k-mers mean k-mers belonging to the aforementioned multiple reference genome k-mer database.

The processor (440) estimates μᵢ as the number rᵢ of reference genome-derived reads located in the analysis region of de novo structural variation candidate region i.

The processor (440) may calculate the prior probability VAF_{prior_i} of variant allele frequency using the formula of Equation 2.

The processor (440) may predict the number di of discordant read pairs located in the analysis region of de novo structural variation candidate region i using the prediction model. The processor (440) may predict the number di of discordant read pairs by inputting μᵢ, the length Li of de novo structural variation candidate region i, the number of split reads Sᵢ, and the prior probability of variant allele frequency VAF_{prior_i} into the prediction model.

The processor (440) may calculate the estimated variant allele frequency eVAFᵢ using the formula of Equation 3.

If eVAFᵢ<0.3, the region is treated as a somatic structural variation and excluded. f eVAFᵢ>0.7, the region is treated as an error and excluded. If 0.3≤eVAFᵢ≤0.7, the region is retained as a de novo candidate.

If 0.3≤eVAFᵢ≤0.7, the processor (440) may select de novo structural variation candidate region i as the final de novo structural variation. In other words, if 0.3≤eVAFᵢ≤0.7, the processor (440) may analyze de novo structural variation candidate region i as a germline-derived de novo structural variation candidate.

If the selected de novo structural variation candidate region i is a parent-derived structural variation, the processor (440) may remove it from the final de novo structural variation candidate region.

The processor (440) may evaluate whether to remove the region from the candidate group by performing the same process as in FIG. 3 for each de novo structural variation candidate region.

The processor (440) may generate a clinical report for the subject (child) based on the detected de novo structural variation results. At this time, the clinical report may include variation position, variation type, list of corresponding genes, and disease occurrence risk. Disease occurrence risk means the risk or probability of disease occurrence based on structural variations. Disease occurrence risk may be determined according to previous research results on structural variations. Medical staff may conduct clinical evaluation or establish treatment plans for the subject based on the clinical report.

Methods according to embodiments described in the specification of the present disclosure may be implemented in the form of hardware, software, or a combination of hardware and software.

When implemented in software, a computer-readable storage medium storing one or more programs (software modules) may be provided. One or more programs stored in the computer-readable storage medium are configured for execution by one or more processors in an electronic device. The one or more programs include instructions that cause the electronic device to execute the methods according to the embodiments described in the specification of the present disclosure.

In addition, the de novo structural variation detection method as described above may be implemented as a program (or application) including executable algorithms that may be executed on a computer. The program may be stored and provided on a transitory or non-transitory computer readable medium.

The non-transitory computer readable medium refers to a medium that stores data semi-permanently (e.g., the storage device) and is capable of being read by a device, rather than a medium that stores data for a short period of time, such as a register, cache, or memory. Specifically, the various applications or programs described above may be provided by being stored in the non-transitory computer readable medium such as a CD, a DVD, a hard disk, a Blu-ray disk, a USB, a memory card, a read-only memory (ROM), a programmable read only memory (PROM), an erasable PROM (EPROM), an electrically EPROM (EEPROM), or a flash memory.

The transitory computer readable medium refers to various types of RAM such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct Rambus RAM (DRRAM).

Various examples and aspects of the present disclosure are described below. These are provided as examples, and do not limit the scope of the present disclosure.

The description herein has been presented to enable any person skilled in the art to make, use and practice the technical features of the present disclosure, and has been provided in the context of one or more particular example applications and their example requirements. Various modifications, additions and substitutions to the described embodiments will be readily apparent to those skilled in the art, and the principles described herein may be applied to other embodiments and applications without departing from the scope of the present disclosure. The description herein and the accompanying drawings provide examples of the technical features of the present disclosure for illustrative purposes. In other words, the disclosed embodiments are intended to illustrate the scope of the technical features of the present disclosure. Thus, the scope of the present disclosure is not limited to the embodiments shown, but is to be accorded the widest scope consistent with the claims. The scope of protection of the present disclosure should be construed based on the following claims, and all technical features within the scope of equivalents thereof should be construed as being included within the scope of the present disclosure.

What is claimed is:

1. A method for analyzing a sample of a subject based on de novo structural variation, comprising:
acquiring genome sequencing data of a target individual;
extracting k-mer data of the target individual from the genome sequencing data;
comparing a reference genome k-mer database with the k-mer data of the target individual to select target individual-specific k-mers;
determining the target individual-specific sequencing reads based on the target individual-specific k-mers;
determining de novo structural variation candidate regions based on the target individual-specific sequencing reads;
selecting a final de novo structural variation region from the de novo structural variation candidate regions based on the number of discordant read pairs and variant allele frequency for each of the de novo structural variation candidate regions; and
generating a diagnostic report for the target individual based on the final de novo structural variation region,
wherein the reference genome k-mer database includes k-mer data of the target individual's parents and pan-genome k-mer data.

2. The method of claim 1, wherein the selecting the final de novo structural variation region comprising:
excluding, from the final de novo structural variation region, any candidate region determined to be a parental structural variation.

3. The method of claim 1, wherein the process of predicting the number of discordant read pairs for a candidate region among the de novo structural variation candidate regions comprises:
inputting variables for the candidate region into a pre-trained machine learning model to predict discordant read pairs for the candidate region,
wherein the input variables include an average frequency of reference genome k-mers for an analysis region around the breakpoint of the candidate region, the number of split reads located in the analysis region, the length of the candidate region, and a prior probability of variant allele frequency.

4. The method of claim 3, wherein the step of selecting the final de novo structural variation region comprises:
calculating an estimated variant allele frequency based on the average frequency of the reference genome k-mers, the number of split reads, and the number of discordant read pairs; and
determining the candidate region as the final de novo structural variation region when the estimated variant allele frequency of the candidate region is within a certain range.

5. The method of claim 3, wherein the split read having one end aligned to a breakpoint defining the candidate region.

6. The method of claim 3, wherein the average frequency of the reference genome k-mers is determined by the following equation:
$${μ}_{i} = \frac{{\sum }_{i}^{n} ( {k}_{i} \times {m}_{i} )}{{w}_{i}}$$
(where μᵢ is the average k-mer frequency of the reference genome in de novo structural variation candidate region i, kᵢ is the k-mer frequency of the input sample located in the analysis region, mᵢ is the mappability score of the reference genome, and wᵢ is the length of the analysis region).

7. The method of claim 3, wherein the prior probability of variant allele frequency is determined by the following equation:
$${VAF}_{prio {r}_{i}} = \frac{{w}_{i} \times {s}_{i}}{{\sum }_{j}^{n} ( {k}_{j} \times {m}_{j} ) + {w}_{i} \times {s}_{i}}$$
(where VAF_{prior_i} is the prior probability of variant allele frequency for de novo structural variation candidate region i, kⱼ is the k-mer frequency of the input sample located in the analysis region, mⱼ is the mappability score of the reference genome, wᵢ is the length of the analysis region, and sᵢ is the number of split read located in the analysis region).

8. The method of claim 4, wherein the estimated variant allele frequency is determined by the following equation:
$${eVAF}_{i} = \frac{{s}_{i} + {d}_{i}}{{μ}_{i} + {s}_{i} + {d}_{i}}$$
(where μᵢ is the average k-mer frequency of the reference genome in de novo structural variation candidate region i, sᵢ is the number of split read located in the analysis region, and di is the number of discordant read pairs).

9. The method of claim 4, further comprising:
determining the candidate region as a somatic structural variation when the estimated variant allele frequency of the candidate region is less than a lower threshold, and
determining the candidate region as an error when the estimated variant allele frequency of the candidate region exceeds an upper threshold.

10. A hardware apparatus for detecting de novo structural variations, comprising:
an input device configured to receives genome sequencing data of a target individual who is an analysis target and genome sequencing data of parents of the target individual;
a storage device configured to store a prediction model that predicts the number of discordant read pairs in a de novo structural variation candidate region; and
a processor configured to extract k-mer data of the target individual and the parents from the respective genome sequencing data; build a reference-genome k-mer database including the parents' and pan-genome k-mers; select target-individual-specific k-mers by comparing the database with the target individual's k-mers; determine candidate regions based on target-individual-specific sequencing reads; and select a final de novo structural variation region by filtering out noise from the candidate regions.

11. The hardware apparatus of claim 10, wherein the processor:
determines an average frequency of reference genome k-mers in an analysis region around a breakpoint in a candidate region among the de novo structural variation candidate regions, the number of split reads located in the analysis region, the number of discordant read pairs, and a prior probability of variant allele frequency,
calculates an estimated variant allele frequency based on the average frequency of the reference genome k-mers, the number of split reads, and the number of discordant read pairs, and
determines the candidate region as the final de novo structural variation when the estimated variant allele frequency falls within a certain range by comparing with a threshold value.

12. The hardware apparatus of claim 11, wherein the processor inputs the average frequency of the reference genome k-mers, the number of split reads located in the analysis region, the length of the candidate region, and the prior probability of variant allele frequency into the prediction model to predict the number of discordant read pairs located in the analysis region.

13. The hardware apparatus of claim 11, wherein the split read having one end aligned to a breakpoint defining the candidate region.

14. The hardware apparatus of claim 11, wherein the processor:
determines the candidate region as a somatic structural variation when the estimated variant allele frequency of the candidate region is less than a lower threshold, and
determines the candidate region as an error when the estimated variant allele frequency of the candidate region exceeds an upper threshold.

15. The hardware apparatus of claim 11, wherein the processor excludes, from the final de novo structural variation candidate region, any candidate region determined to be a parental structural variation.
